(19) United States
(12) Patent Application Publication
(10) Pub. No.: US 2013/0280190 A1
(43) Pub. Date: Oct. 24, 2013

(54) DIETHYLHEXYL BUTAMIDO TRIAZONE WITH HIGH PURITY, AND PROCESS FOR ITS PREPARATION

(71) Applicants: Francesco Maestri, Bergamo (IT); Luca Bemporad, Bergamo (IT); Ferruccio Berte', Bergamo (IT)

(72) Inventors: Francesco Maestri, Bergamo (IT); Luca Bemporad, Bergamo (IT); Ferruccio Berte', Bergamo (IT)

(73) Assignee: 3V Sigma S.p.A., Milano (IT)

(21) Appl. No.: 13/492,464

(22) Filed: Jun. 8, 2012

(30) Foreign Application Priority Data
Apr. 18, 2012 (IT) MI2012A000645

(51) Int. Cl. A61K 8/49; A61Q 17/04; C07D 251/70

(52) U.S. Cl. 424/60; 544/197

(57) ABSTRACT

Disclosed is the compound of formula

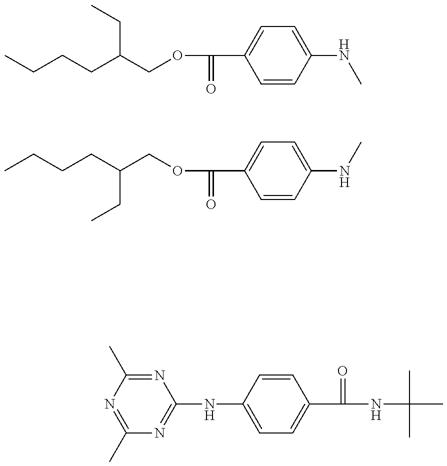

known by the name of diethylhexyl butamido triazone, characterised by a low content of impurities deriving from the preparation process, and by low APHA colour values. The compound according to the invention is obtained by reacting cyanuryl chloride with one equivalent of para-aminobenzoic acid, reacting the intermediate obtained with two equivalents of 2-ethylhexyl-4-aminobenzoate, activating the carboxy group, for example by formation of acyl chloride, and final reaction with tert-butylamine.

The compound according to the invention is a UV-B filter which can be advantageously used in cosmetic formulations.

### CROSS-REFERENCE TO RELATED APPLICATIONS

This application claims priority to and the benefit of Italian Application No. MI2012A000645 filed on Apr. 18, 2012, the content of which is incorporated herein by reference in its entirety.

The present invention relates to diethylhexyl butamido triazone (INCI name: diethylhexyl butamido triazone), characterised by a 2-ethylhexyl-4-aminobenzoate content ranging from 1 to 1000 ppm and by an APHA colour of between 0 and 400, preferably between 0 and 250.

The invention also relates to the preparation process of said compound.

### PRIOR ART

Diethylhexyl butamido triazone or benzoic acid, 4,4′-[[6-[[4-[[(1,1-dimethyl-ethyl)amino]carbonyl]phenyl]amino-1,3,5-triazin-2,4-diyl]diimino]-bis-,-bis(2-ethylhexyl) ester, of formula:

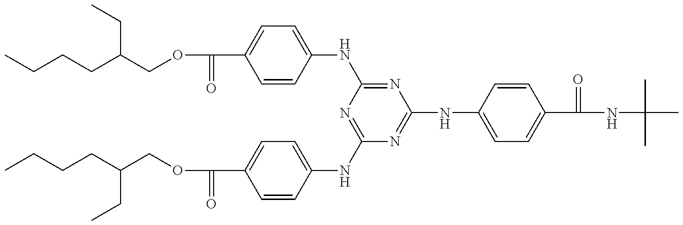 [SMILES: CCCCC(CC)COC(=O)c1ccc(Nc2nc(Nc3ccc(C(=O)NC(C)(C)C)cc3)nc(Nc3ccc(C(=O)OCC(CC)CCCC)cc3)n2)cc1]

is a known UV-B filter used in particular for applications in the cosmetics industry, which is available on the market under the UVASORB HEB® brand.

The synthesis of diethylhexyl butamido triazone was disclosed in U.S. Pat. No. 5,346,691, and involves the following preparation scheme:

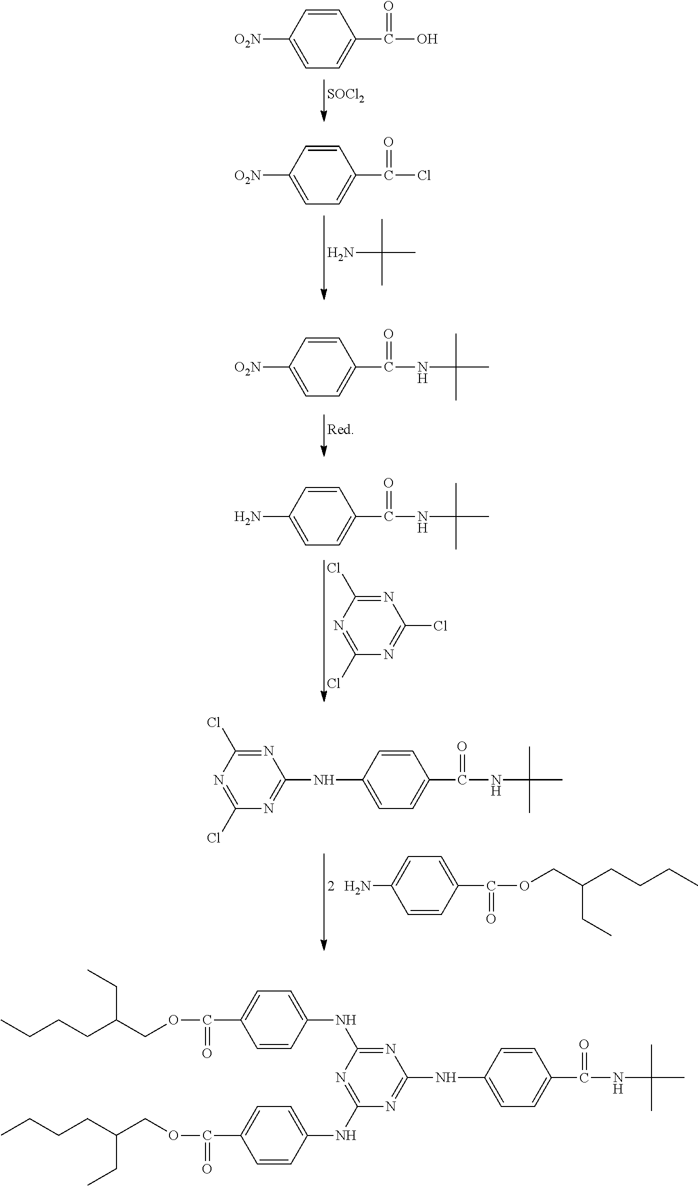

The process involves reacting cyanuryl chloride with one equivalent of para-amino benzoic acid tert-butylamide, previously prepared from 4-nitro-benzoic acid. The mono-substituted triazine obtained is then reacted with two equivalents of 2-ethylhexyl-4-aminobenzoate to give the desired compound.

Said process has a number of disadvantages, associated with the large number of steps involved and the need to purify the various intermediates often in order to guarantee satisfactory purity of the final product. The preparation of para-amino benzoic acid tert-butylamide from 4-nitro-benzoic acid is particularly laborious, and requires a number of steps. The overall efficiency of the process is therefore affected, with adverse effects on the costs of production. Moreover, the product obtained has characteristics which could be improved in terms of purity and of other properties important for the purpose of their use, such as colour, odour and stability. This may be partly due to the fact that it is almost impossible to obtain a product with a low content of the intermediate 2-ethylhexyl-4-aminobenzoate, used in the last step. A residual amount of this substance in the end product is therefore inevitable and difficult to eliminate, partly due to the difficulty of purifying the end product. The presence of this amine impurity is undesirable in view of the residual reactivity of the amine group, and its low molecular weight.

There is therefore a need for a novel form of diethylhexyl butamido triazone with a low content of amine impurities that better meets the stringent formulation requirements in particular sectors such as the cosmetics industry.

### DESCRIPTION OF THE INVENTION

It has now been found that diethylhexyl butamido triazone can be obtained in a form characterised by a 2-ethylhexyl-4-aminobenzoate content of between 1 ppm and 1000 ppm, and an APHA colour of between 0 and 400, preferably between 0 and 250. Said form, which was not obtainable by the known process, even after repeated purifications of the end product, constitutes a first subject of the invention.

The novel product has in particular a 2-ethylhexyl-4-aminobenzoate content of between 1 and 1000 ppm, preferably between 1 and 700 ppm, and more preferably between 1 and 400 ppm. The impurity content can be determined by the HPLC method, using a Phenomenex Luna C8 reverse-phase column, length 150 mm, inner diameter 4.6 mm and particle size 5 μm, as stationary phase, and a mixture of two eluents, mixed in gradient mode, as mobile phase: the first corresponds to an aqueous solution of sodium dihydrogenphosphate (0.005M) at a pH of approx. 4.5, and the second to methanol. The eluent flow rate is 1.5 ml/minute. A UV-visible spectrophotometer with signal acquisition at 300 nm was used as detector. The product according to the invention has a purity exceeding 98.5%, whereas the product according to the prior art usually has a purity of approx. 97.5%.

The APHA colour was determined according to the ASTM D1209 “Standard Test Method for Color of Clear Liquids (Platinum-Cobalt Scale)”. The APHA value was measured on a solution of diethylhexyl butamido triazone dissolved in toluene at the concentration of 10% weight/volume.

The APHA colour is a very important parameter for the purpose of applications in certain fields: the lower the APHA value, the better the colorimetric characteristics of the product tested. In particular a low APHA colour value corresponds to a substantially colourless product, which does not impart undesirable colouring or dominants to formulations containing said product.

The diethylhexyl butamido triazone according to the invention can therefore be advantageously introduced into formulas for cosmetics, either as a single sunscreen or in combination with other known sunscreens, as a replacement for the one obtained by the known process. These formulations constitute a second object of the invention. Said formulations will preferably contain one or more conventional UV-A and UV-B sunscreens such as those listed in ANNEX VII to the European Cosmetics Directive (76/768/EEC). Even more preferably, the formulations may contain, in addition to the sunscreen according to the invention, one or more sunscreens selected from: 2-ethylhexyl p-methoxycinnamate, 2-hydroxy-4-methoxybenzophenone, 2-hydroxy-4-methoxybenzophenone-5-sulphonic acid, 3-(4′-methylbenzylidene)-d,l-camphor, 2,4,6-trianiline-(p-carbo-2′-ethylhexyl-1′-oxy)-1,3,5-triazine, 4-(tert-butyl)-4′-methoxy-dibenzoylmethane, 2-cyano-3,3-diphenylacrylic acid 2-ethylhexyl ester, bis-ethylhexyloxyphenol-methoxy-phenyl-triazine, methylene-bis-benzotriazolyl-tetramethylbutylphenol, benzoic acid 2-(4-diethylamine-2-hydroxybenzoyl)-hexyl ester, titanium dioxide, and zinc oxide.

The novel form of diethylhexyl butamido triazone is obtained by a novel process which constitutes a further subject of the invention. Said process is illustrated in the scheme below:

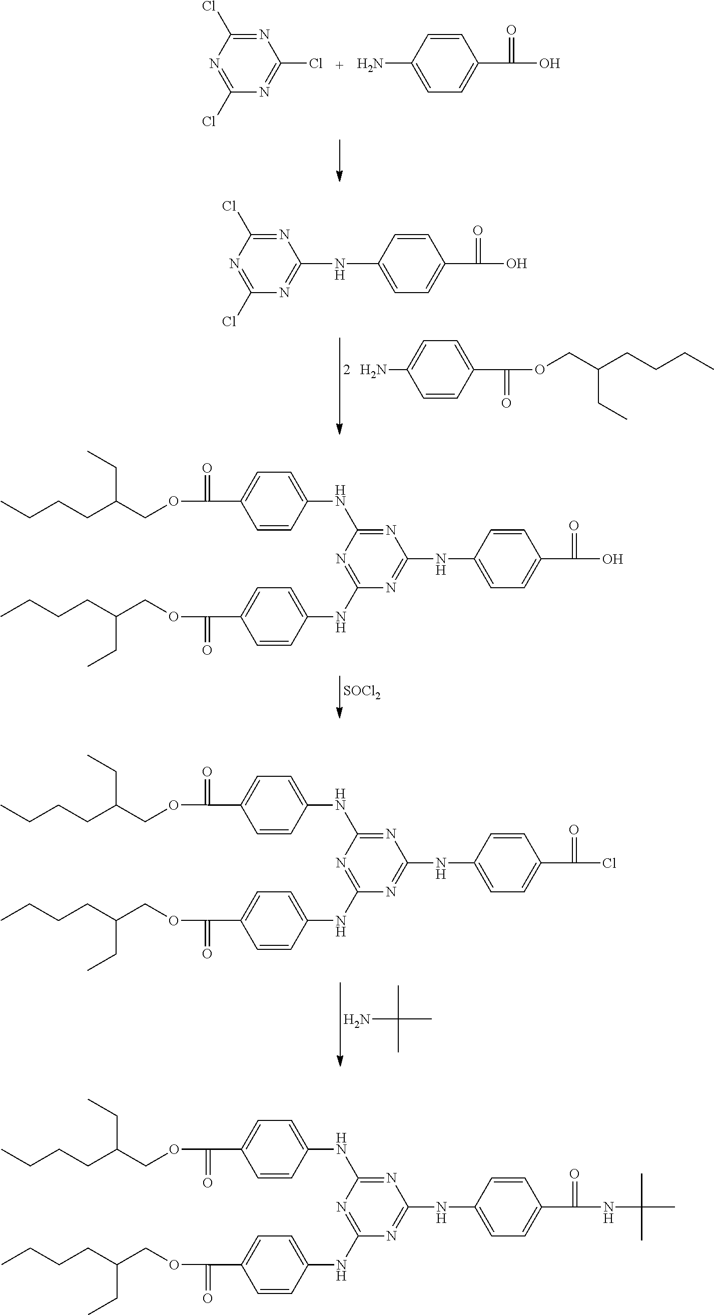

The process comprises:

a) reacting cyanuryl chloride with one equivalent of para-amino-benzoic acid to give a compound of formula:

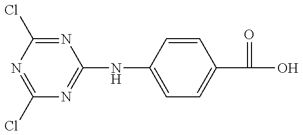 [SMILES: O=C(O)c1ccc(Nc2nc(Cl)nc(Cl)n2)cc1]

or a salt thereof, in particular an alkali metal salt, preferably the sodium salt;

b) reacting the compound obtained in a) with two equivalents of 2-ethylhexyl-4-aminobenzoate

to give a compound of formula:

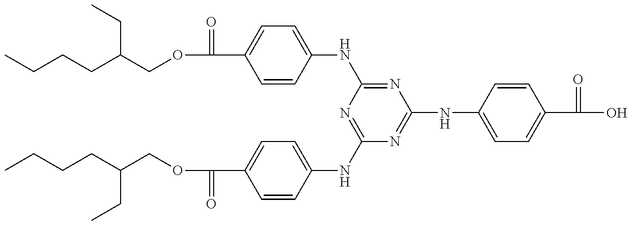 [SMILES: CCCCC(CC)COC(=O)c1ccc(Nc2nc(Nc3ccc(C(=O)O)cc3)nc(Nc3ccc(C(=O)OCC(CC)CCCC)cc3)n2)cc1]

or a salt thereof, in particular an alkali metal salt, preferably the sodium salt;

c) reacting the compound obtained in b) with agents selected from halogenating agents such as thionyl chloride, sulphuryl chloride, phosphorus trichloride, phosphorus pentachloride, phosphorus oxychloride, oxalyl chloride, phosgene or mesyl or tosyl halides and subsequent reaction with tert-butylamine.

The equivalent cyanuryl bromide could obviously be used instead of cyanuryl chloride.

The order of steps a) and b) can be reversed, reacting the cyanuryl halide first with two equivalents of 2-ethylhexyl-4-aminobenzoate and then with one equivalent of para-amino-benzoic acid.

The order of steps b) and c) can be reversed, reacting the compound obtained in step a) with halogenating agents or with mesyl or tosyl halides and then with tert-butylamine, under conditions that safeguard the cyanuryl active chlorine atoms, and then reacting the product thus obtained with two equivalents of 2-ethylhexyl-4-aminobenzoate.

The reaction between cyanuryl halide and para-amino-benzoic acid is conducted in a solvent, in the presence of a base, which can also be added at a later stage, and in such a way as to promote the formation of the product of mono-substitution.

The preferred solvents are all solvents compatible with the reagents, in particular ketones such as acetone, methyl ethyl ketone, methyl isobutyl ketone and diisobutylketone, aromatic hydrocarbons such as benzene, toluene and xylene, saturated hydrocarbons such as pentane, hexane, cyclohexane, heptane, octane, isooctane, decane and isoparaffin, halogenated solvents such as methylene chloride, chloroform, dichloroethane and trichloroethane, ethers such as diethylether, tetrahydrofuran and dioxane, esters such as methyl acetate or ethyl acetate, nitriles such as acetonitrile, and alcohols such as isopropanol and tert-butanol. More preferred solvents are ketones, acetone being particularly preferred. Said solvents are preferably anhydrous, but can also contain small amounts of water.

Suitable bases are oxides, hydroxides, alkaline or alkaline earth carbonates or bicarbonates, preferably sodium hydroxide, sodium carbonate and sodium or potassium bicarbonate or tertiary amines, preferably trimethylamine, triethylamine or pyridine.

The reaction can be conducted at temperatures between −30° C. and 60° C., preferably between −20° C. and 30° C., and even more preferably between −10° C. and 10° C., and at pressures of between 0.01 bar and 10 bar, preferably between 0.1 bar and 2 bar, and even more preferably between 0.5 and 1.5 bar.

The stage b) reactions are preferably conducted in a solvent compatible with the reagents, but in some cases the solvent function can be performed by an adequate excess of the reagent.

The preferred solvents are all solvents compatible with the reagents, in particular ketones such as acetone, methyl ethyl ketone, methyl isobutyl ketone and diisobutylketone, aromatic hydrocarbons such as benzene, toluene and xylene, saturated hydrocarbons such as pentane, hexane, cyclohexane, heptane, octane, isooctane, decane and isoparaffin, halogenated solvents such as methylene chloride, chloroform, dichloroethane and trichloroethane, ethers such as diethylether, tetrahydrofuran and dioxane, esters such as methyl acetate and ethyl acetate, nitriles such as acetonitrile, and alcohols such as isopropanol and tert-butanol. More preferred solvents are aromatic hydrocarbons, toluene and xylene being particularly preferred.

Said solvents are preferably anhydrous, but can also contain small amounts of water.

The reaction can be conducted in the presence of a base, which can also be added at a later stage. Suitable bases are oxides, hydroxides, alkaline or alkaline earth carbonates or bicarbonates, preferably sodium hydroxide, sodium carbonate and sodium or potassium bicarbonate or tertiary amines, preferably trimethylamine, triethylamine or pyridine.

The reaction can be conducted at temperatures between 10° C. and 220° C., preferably between 30° C. and 200° C. and even more preferably between 50° C. and 160° C., and at pressures of between 0.01 bar and 10 bar, preferably between 0.1 bar and 2 bar, and even more preferably between 0.5 and 1.5 bar.

Finally, the carboxy group can be converted to the COX group (stage c) by known organic chemistry methods (A. I. Vogel, “Chimica organica pratica”, Casa Editrice Ambrosiana, Third edition, 1967; M. B. Smith and J. March, “March's Advanced Organic Chemistry”, Wiley, Sixth edition, 2007), preferably by treatment with thionyl chloride, sulphuryl chloride, phosphorus trichloride, phosphorus pentachloride, phosphorus oxychloride, oxalyl chloride, phosgene, and even more preferably with thionyl chloride.

The reaction can be conducted in the presence of a solvent, but in some cases the solvent function can be performed by an adequate excess of the halogenating agent.

The preferred solvents are all solvents compatible with the reagents and with the product of reaction, and in particular with the halogenating agent and the COX group which is synthesised. Aromatic hydrocarbons such as benzene, toluene and xylene, saturated hydrocarbons such as pentane, hexane, cyclohexane, heptane, octane, isooctane, decane and isoparaffin, halogenated solvents such as methylene chloride, chloroform, dichloroethane and trichloroethane, ethers such as diethylether, tetrahydrofuran and dioxane, esters such as methyl acetate and ethyl acetate, nitriles such as acetonitrile, and ketones can be used in particular. The preferred solvent is an aromatic hydrocarbon, in particular toluene or xylene, more preferably xylene. The same solvent as used for stage b) is preferably used.

The reaction can be conducted at temperatures between 10° C. and 150° C., preferably between 30° C. and 100° C. and even more preferably between 50° C. and 80° C., and at pressures of between 0.01 bar and 10 bar, preferably between 0.1 bar and 2 bar, and even more preferably between 0.5 and 1.5 bar.

The processes according to the invention are advantageous because they can also be performed using a single solvent, and with no need to isolate and/or purify the intermediates. Solvents of a different nature can also be used if necessary, and isolations and intermediate purifications can optionally be introduced.

The process according to the invention is more advantageous than the known method, wherein the reaction with 2-ethylhexyl-4-aminobenzoate constituted the last stage of synthesis, because the reaction with 2-ethylhexyl-4-aminobenzoate is brought forward to the first or second stage of synthesis, so the residual content of that intermediate in the end product can be considerably reduced or eliminated. The product also possesses improved organoleptic characteristics, in particular as regards odour, which is substantially neutral or absent.

The novel process consists of a smaller number of steps, does not require onerous reactions such as reduction, in particular hydrogenation of nitroderivatives, and is therefore more efficient.

The invention is illustrated in greater detail in the examples below.

### Example 1

### Synthesis of 4-((4,6-bischloro-1,3,5-triazin-2-yl)amino)benzoic acid

75.0 g of cyanuryl chloride, 37.3 g of sodium bicarbonate and 304 g of anhydrous acetone, precooled to −10° C., were loaded into a 2-litre flask fitted with a stirrer, thermometer, condenser and dropping funnel.

A solution, precooled to −10° C., consisting of 54.7 g of p-aminobenzoic acid and 523 g of anhydrous acetone, was added in 45 min, under stirring at −10° C.

After 60 minutes' stirring at −10° C., 100 g of demi water, precooled to 0-2° C., was added in approx. 15 minutes. After two more hours of completion at −10° C. the product, in the form of a white solid in suspension, was isolated by filtration under vacuum.

The wet filtration panel was then washed in sequence, first with aqueous acetone and then with anhydrous acetone. The wet panel was stove-dried under vacuum to obtain 139 g of fine white powder, consisting of a mixture of the desired product and inorganic salts. The powder was analysed, determining an active chlorine content of 19.8% w/w as the difference between total chlorine (29.4% w/w) and free chlorides (9.6% w/w).

The product was also characterised by UPLC-MS chromatography.

### Example 2

### Synthesis of 4-(4,6-bis(4-((2-ethylhexyloxy)-carbonyl)phenylamine)-1,3,5-triazin-2-ylamino)benzoic] acid

568 g of anhydrous xylene and 139.0 g of the product prepared in example 1 were loaded into a 2-litre flask fitted with a stirrer, thermometer, dropping funnel and condenser.

645 g of a 30% xylene solution of 2-ethyl hexyl 4-aminobenzoate was added in 30 minutes to the mixture, stirred at 90° C. When the addition had been completed, the mixture was maintained at 90° C. for 15 min and then heated to 125° C. in 60 minutes, obtaining a thick whitish suspension. The mixture was maintained under stirring at 125° C. for 3 hours, during which time a gradual reduction in the development of hydrochloric acid and an increase in the fluidity of the mixture was observed. After cooling to 80-90° C., 280 g of 15% aqueous sodium carbonate was added cautiously. After 30 minutes' mixing at 70-80° C., stirring was interrupted and the underlying alkaline aqueous phase was discharged. After two further aqueous washes, the residual water was removed by azeotropic distillation, followed by distillation of xylene to concentrate the solution.

680 g of whitish dispersion was obtained, containing approx. 275 g of the desired product, which was characterised by UPLC-MS. The dispersion “as is” was used for the subsequent stages of synthesis.

### Example 3

### Synthesis of 4-(4,6-bis(4-((2-ethylhexyloxy)-carbonyl)phenylamine)-1,3,5-triazin-2-ylamino)benzoyl chloride)

62.4 g of thionyl chloride, 31.2 g of anhydrous xylene and 0.22 g of dimethylformamide were loaded into a 1-litre flask fitted with a stirrer, thermometer, condenser and dropping funnel. 220 g of the final dispersion obtained in example 2 was fed in 3 hours into the well-stirred mixture, maintained at 70° C.

The hydrochloric acid and sulphur trioxide released during dripping were removed by bubbling in an aqueous solution of sodium hydroxide. When the addition had been completed, the mixture was stirred at 70° C. for a further 2 hours. The excess thionyl chloride was then removed by distillation under vacuum, and the excess xylene as distillation tail. 235 g of xylene mixture, containing approx. 89 g of the desired acyl chloride, remained in the flask. A sample of acyl chloride was isolated for characterisation by complete removal of the solvent. The acyl chloride was reacted with an excess of methanol to obtain the corresponding methyl ester, the structure of which was confirmed by IR, NMR and UPLC-MS tests.

### Example 4

### Synthesis of benzoic acid, 4,4′-[[6-[[4-[[(1,1-dimethyl-ethyl)amino]carbonyl]phenyl]amino-1,3,5-triazin-2,4-diyl]diimino]bis-,-bis(2-ethylhexyl) ester

36.5 g of tert-butylamine was added under stirring, in 15 minutes, to 235 g of the xylene mixture obtained in example 3, maintained at 30-40° C. by cooling. After a further 30 minutes at 40° C., the mixture was heated to 95° C. in 60 minutes.

After 60 minutes at 95° C. the temperature was reduced to 50-60° C., and 212 g of 12.5% w/w sodium carbonate was added under stirring.

After 15 minutes at 50° C., stirring was interrupted, and the underlying alkaline aqueous phase was discharged after separation. The organic phase was washed with water. After removal of the residual water by azeotropic distillation in the presence of a filtrating earth, the mixture was cooled to 60° C. and filtered hot.

The product was then isolated by complete removal of the xylene by distillation under vacuum. The molten product thus obtained was flaked and ground, obtaining 95 g of a whitish powder with a chromatographic purity of 98.65%, APHA colour (10% w/v in toluene)=189, a 2-ethylhexyl-4-aminobenzoate content of 600 ppm, softening point 90-120° C., and extinction E′=1522 at 311 nm. The structure of the product was confirmed by IR, NMR and UPLC-MS tests.

The product obtained with the process according to U.S. Pat. No. 5,346,691 has a purity of 97.55%, APHA colour (10% w/v in toluene)=712 and a 2-ethylhexyl-4-aminobenzoate content of 4800 ppm.

1. Diethylhexyl butamido triazone characterised by a 2-ethylhexyl-4-aminobenzoate content lower than 1000 ppm and by an APHA colour ranging from 0 to 400.

2. Diethylhexyl butamido triazone with a 2-ethylhexyl-4-aminobenzoate content ranging from 1 to 1000 ppm.

3. Diethylhexyl butamido triazone with a 2-ethylhexyl-4-aminobenzoate content ranging from 1 to 700 ppm.

4. Diethylhexyl butamido triazone with an APHA colour (10% sol. in toluene) ranging from 0 to 250.

5. A process for the preparation of diethylhexyl butamido triazone of claim 1 which comprises:
a) reacting cyanuryl chloride with one equivalent of para-amino-benzoic acid to give a compound of formula: 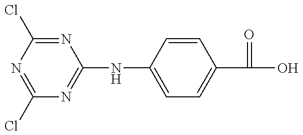 [SMILES: O=C(O)c1ccc(Nc2nc(Cl)nc(Cl)n2)cc1]
or a salt thereof;
b) reacting the compound obtained in a) with two equivalents of 2-ethylhexyl-4-aminobenzoate to give a compound of formula: 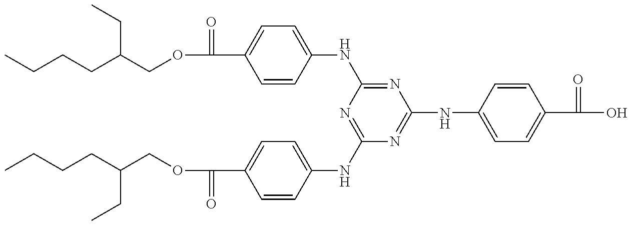 [SMILES: CCCCC(CC)COC(=O)c1ccc(Nc2nc(Nc3ccc(C(=O)O)cc3)nc(Nc3ccc(C(=O)OCC(CC)CCCC)cc3)n2)cc1]
or a salt thereof;
c) reacting the compound obtained in b) with agents selected from halogenating agents, selected from the group consisting of thionyl chloride, sulphuryl chloride, phosphorus trichloride, phosphorus pentachloride, phosphorus oxychloride, oxalyl chloride, and phosgene, or with mesyl or tosyl halides and subsequently with tert-butylamine.

6. A process according to claim 5 wherein step b) precedes step a).

7. A process according to claim 5 wherein the compound obtained from step a) is reacted first with halogenating agents or with mesyl or tosyl halides and then with tert-butylamine, under conditions which safeguard the cyanuryl active chlorine atoms, the resulting product being then reacted with two equivalents of 2-ethylhexyl-4-aminobenzoate.

8. Cosmetic formulations containing the compound of claim 1 as UV-B filters.

9. Cosmetic formulations of claim 8 further comprising additional UV-A and/or UV-B filters, cosmetic active ingredients and suitable excipients.
